(19) United States
(12) Patent Application Publication
(10) Pub. No.: US 2018/0264179 A1
(43) Pub. Date: Sep. 20, 2018

(54) BIODEGRADING IMPLANTABLE OCULAR SUSTAINED RELEASE DRUG DELIVERY SYSTEM

(71) Applicant: Biohealthways, Inc., Brooklyn, NY (US)

(72) Inventors: David PAN, Brooklyn, NY (US); Brian W. PAN, Brooklyn, NY (US)

(21) Appl. No.: 15/924,318

(22) Filed: Mar. 19, 2018

Related U.S. Application Data
(60) Provisional application No. 62/473,458, filed on Mar. 19, 2017.
(60) Provisional application No. 62/537,931, filed on Jul. 27, 2017.
(60) Provisional application No. 62/581,673, filed on Nov. 4, 2017.

(51) Int. Cl. A61L 31/14; A61F 9/00; A61L 31/10; A61L 31/16

(52) U.S. Cl. CPC A61L 31/148; A61F 9/0017; A61L 31/10; A61F 2210/0004; A61L 31/145; A61F 2250/0067; A61F 2230/0069; A61L 31/16

(57) ABSTRACT

An ocular implant is provided for an intraocular delivery of a therapeutic biologic agent. The implant may be used intracamerally or intravitreally. The implant may include a sustained-release biodegradable core and a biodegradable shell, wherein the shell has a longer biodegradable half-life than the core. The core may include a biodegradable gel medium, an active therapeutic biologic agent, and a biologic stabilizer. Upon insertion into the anterior chamber or vitreous body of an eye, the therapeutic biologic agent is released over an extended period, that may range from one day to one year. The therapeutic biologic agent may be, for example, tissue-plasminogen activator, an anti-VEGF agent, or another biopharmaceutical. The biodegradable implant may completely dissolve after implantation and need not be removed.

### CROSS REFERENCE TO RELATED APPLICATIONS

This application claims priority to U.S. Provisional Patent Application No. 62/473,458, filed Mar. 19, 2017, and U.S. Provisional Patent Application No. 62/537,931, filed Jul. 27, 2017, and U.S. Provisional Patent Application No. 62/581,673, filed Nov. 4, 2017.

### FIELD OF THE INVENTION

This invention relates to an intraocular sustained release drug delivery implant system and formulation. In particular, this invention relates to an implant that delivers biopharmaceutical agents for treating an ocular condition, by inserting an implant device into an ocular region or site, wherein a therapeutic biologic agent is released over time and the implant is fully resorbable (biodegradable).

### BACKGROUND

Biologic therapeutics are well known and widely prescribed due to their benefits and efficacy. However, biologic therapeutics (biopharmaceuticals) have several difficulties associated with their use. Biologic therapeutic agents may be associated with undesired side effects at high systemic concentrations. Biologic therapeutic agents generally must be injected (oral delivery is not normally an option), so repeated painful and expensive administration of biologics may be required, causing significant inconvenience and expense. Biologics also tend to be chemically unstable compared to small molecules, making it a challenge to deliver sustained required doses over time. It is therefore desirable to provide controlled sustained release formulations and systems for biologic therapeutic delivery to reduce the systemic exposure and frequency of administration, as well as to improve biologic therapeutic safety and patient compliance.

Mammalian eyes are an organ protected from exogenous substances and external stress by various barriers. Therefore, therapeutic drugs must be transported across several protective barriers regardless of which administration route is utilized, such as eye-drops, and subconjunctival, sub-tenon's and intravitreal injection and/or implant.

For the treatment of the anterior segment of the eye (cornea, conjunctiva, sclera, anterior uvea), usually topical ocular eye-drops are used. In general, topical eye drop application of drugs do not efficiently reach anterior or posterior anatomy of the eye. An eye-drop, irrespective of the instilled volume, often eliminates rapidly within five to six minutes after administration, and only a small amount (1-3%) of an eye-drop reaches the intraocular tissue. Thus, it is difficult to provide and maintain an adequate concentration of drug in the pre-corneal area. More than 75% of applied ophthalmic solution is lost via nasolacrimal drainage and absorbed systemically via conjunctiva, hence ocular drug availability is very low. To increase ocular bioavailability and prolong the retention time on the ocular surface, numerous ophthalmic vehicles such as viscous solutions, suspensions, emulsions, ointments, aqueous gels, and polymeric inserts, have been investigated for topical application to the eye.

The unique anatomy and physiology of the eye and its protective barriers prevent the topical eye-drop-administrated drugs from penetrating into certain target tissues. Therefore, intraocular injections, which can be classified as intracameral or intravitreal, generally improve ocular drug availability by being proximal and inside the blood-ocular barriers. Intracameral injections can treat areas which include the anterior eye anatomy (e.g. trabecular meshwork, Schlemm's canal). Intravitreal injections can treat the posterior eye anatomy (e.g. retina, vitreous, choroid). Subretinal injections can treat areas under the retina. Therefore, clinical therapeutics can be administered, periocularly (e.g. topical eye drops) or intraocularly (e.g. intracameral or intravitreal injections, among other injection methods). Currently, there is also rapidly growing interest in drug delivery systems (DDS's) to the intraocular space (anterior and posterior) segments of the eye.¹ This trend is toward a polymeric implant system implanted or injected directly into the vitreous, to obtain long-term, sustained release of drugs. Limited examples of anterior placement of a polymeric implant are available. In addition, limited biodegradable (fully resorbable) intraocular polymeric implants examples are described.

A significant issue with treating ocular diseases is that compliance with therapeutic regimens is problematic, particularly among patients who have chronic diseases such as glaucoma (open- or closed-angle types), and refractory chorio-retinal diseases, including uveitis, macular edema, neovascular (wet) and atrophic (dry) age-related macular degeneration (AMD), and retinitis pigmentosa (RP). It has been reported that fewer than 25% of patients use their eye drops continuously for 12 months.² Similarly, for the treatment of neovascular AMD and macular edema secondary to retinal vein occlusion (RVO), the standard therapy is intravitreal injections of an anti-vascular endothelial growth factor (VEGF) monoclonal antibody fragment once a month. But studies suggest that ˜20% of patients discontinue treatment within one year, and ˜40% of patients discontinue within two years.³ The monthly cost could be thousands of dollars, so that means effective treatment faces a serious social problem. In addition, frequent intravitreal injections can cause complications, such as increased intraocular pressure,⁴ endophthalmitis, and retinal detachment. Therefore, new methods of treatment of chronic eye diseases is urgently needed. ¹ See for example Yuhua Weng, et al., “Nanotechnology-based strategies for treatment of ocular disease,” Acta Pharmaceutica Sinica B, Available online 18 Nov. 2016, http://(dx.doi.org/10.1016/j.apsb.2016.09.001. (http://www.sciencedirect.com/science/article/pii/S2211383516300855)² G. Shafranov, “Glaucoma Therapy: Compliance, Adherence, Persistence, and Alliance,” Glaucoma Today, July/August 2006, 40-43, http://glaucomatoday.com/2006/08/0706_04.html/³ J. Stokes and A. Chang, “Adherence and Persistence with anti-VEGF treatment for AMD,” MIVision, 27 Jun. 2016, http://www.mivision.com.au/adherence-and-persistence-with-anit-vegf-treatment-for-amd/; M. Duffy, “Which Real-Life Factors Influence Adherence to Lucentis Treatment for Macular Degeneration?” http://www.visionaware.org/blog/visionaware-blog/which-real-life-factors-influence-adherence-to-lucentis-treatment-for-macular-degeneration/12⁴ Brennan D Eadie, et al., “Association of Repeated Intravitreous Bevacizumab Injections With Risk for Glaucoma. Surgery,” JAMA Ophthalmol, Published online Mar. 16, 2017; doi:10.1001/jamaophthalmol.2017.0059

By delivering a drug directly into the vitreous cavity, blood-ocular barriers can be circumvented, and intraocular therapeutic levels can be achieved with reduced risk of systemic toxicity. This route of administration typically results in a short half-life unless the drug can be delivered using a formulation capable of providing sustained release.

Consequently, a biodegradable implant for delivering a therapeutic agent to an ocular region may provide significant medical benefit for patients afflicted with a medical condition of the eye. There are multiple competing ocular delivery routes for anterior and posterior eye diseases. These include systemic administration, topical eye drops, topical injections (intravitreal solution/suspension, intravitreal implant, periocular), and nanosystems. Topical eye drops have efficacy for anterior eye diseases, but the residency time and bioavailable payload delivered to the affected area may not be sufficient for certain drugs. Topical periocular administration is not invasive but is inefficient in prolonged drug retention time. Intraocular solutions/suspensions are effective topical treatments to the back of eye, but residency time is an issue. This invention relates to a biodegrading intravitreal implant using a related nanosystem to control release, which is different from conventional approaches.

Regarding topical eye drops, many topical eye drops have been formulated to treat a wide variety of ocular diseases, but cannot provide drug delivery intraocularly. These include topical anti-inflammatory steroids drops, antibacterial/fungal drops, antiviral drops, glaucoma eye drop treatments, pain treatments, and others. A number of the drugs in eye drops are being or have been formulated for delivery intravitreously or intracamerally. Continued research for eye drops include longer lasting formulations or drops with molecules small enough to permeate the ocular anatomical barriers.

Other periocular drug administration methods include iontophoresis, light-activated therapies (Visudyne), subconjunctival injection, punctal plugs, and others. There is varying efficacy of these administration routes. While less invasive, these methods also require frequent administration and potentially high dosages with concomitant toxicity. For example, using iontophoresis for back-of-eye diseases requires the drug to traverse multiple ocular barriers, but requires specialized equipment and someone to supervise administration. For subconjunctival injections or punctal plugs, the drug faces the same issues of traversing multiple ocular barriers, meaning dosing will be low.

Intraocular injections have been developed for introducing biologics inside the eye, with limited ability to sustain delivery over periods of time up to about two months.⁵ Two major indications where biologics may be used are for: 1) neovascularization in the posterior segment of the eye, and 2) glaucoma. Intraocular injections can be delivered in liquid aqueous form (as in ranibizumab), in liquid hydrogel form (as in aflibercept), in nanosystems or other embodiments, and in implants (biodegrading or non-biodegrading). The location of these injections may vary on which ocular anatomy to localize drugs to. For glaucoma, intracameral or intravitreal implantations are both practical options, because the drainage path in the eye will pass from the posterior to the anterior portion of the eye. For neovascularization in the eye, usually intravitreal implantation makes sense since the retinal is at the back (posterior) of the eye. Subretinal or submacular injections may be useful to localize protein delivery, gene therapies and stem cells, or other advanced biologics.^{6, 7} ⁵ Yuhua Weng, et al., “Nanotechnology-based strategies for treatment of ocular disease,” Acta Pharmaceutica Sinica B, Available online 18 Nov. 2016, http://dx.doi.org/10.1016/j.apsb.2016.09.001. (http://www.sciencedirect.com/science/article/pii/S2211383516300855)⁶ Kumar A, et al., “Subretinal Drug Delivery in Wet Age Related Macular Degeneration with Sub-macular Hemorrhage.” DJO 2014; 25:99-102 http://dx.doi.org/10.7869/djo.87⁷ Haupert, Christopher L et al., “Pars plana vitrectomy, subretinal injection of tissue plasminogen activator, and fluid-gas exchange for displacement of thick submacular hemorrhage in age-related macular degeneration,” American Journal of Ophthalmology, Volume 131, Issue 2, 208-215; http://dx.doi.org/10.1016/50002-9394(00)00734-0

For neovascularization (age- or diabetes-related) in the posterior segment of the eye, this indication is now treated with aqueous anti-VEGF biologics/VEGF-trap or aptamer proteins injected intravitreously: pegaptanib (Macugen), aflibercept (Eylea), bevacizumab (Avastin), and ranibizumab (Lucentis).⁸ Neovascularization can occur in diabetic eyes or in aging eyes when new microvascular networks form, in response to poor local perfusion (poor blood circulation) or ischemia (poor blood supply). These microvascular networks cause vision loss when blood leaks or causes retinal swelling. Anti-VEGF drugs have helped improve visual acuity by preventing further vision loss; however, the stability of these biologics pose a challenge due to their short half-lives in the vitreous. Genentech/Roche/Novartis have achieved ˜1 month delivery of Lucentis in the eye by delivering the drug in an aqueous solution. Note that Avastin is not approved for use in the eye but also lasts ˜1 month. Regeneron/Biolex have achieved ˜2 month sustained delivery of aflibercept (Eylea) by using a polymer hydrogel liquid suspension with physical cross-links such as PEG (polyethylene glycol) multi-blocked with butylene terephthalate. A potential takeaway from comparing Eylea vs. Lucentis approaches is that topical intravitreal injections utilizing polymer hydrogel are a form of protection for the therapeutic agent from the intravitreal environment. Another takeaway is that biologics tend to have rather short half-lives, which make sustained delivery intraocularly a challenge. ⁸ Mary Joseph, et al., “Recent perspectives on the delivery of biologics to back of the eye,” Expert Opinion on Drug Delivery, Published online: 6 Sep. 2016; http://dx.doi.org/10.1080/17425247.2016.1227783

Subretinal or submacular injections may be useful to localize protein delivery, gene therapies and stem cells, or other advanced biologics. Accessing the subretinal space or delivering therapy in a localized fashion has benefits. For example, submacular injections may be useful in displacing submacular hemorrhages when the delivered biologic is recombinant tissue plasminogen activator (r-tPA).⁷ Delivery of r-tPA and an anti-VEGF such as bevacizumab (Avastin), could have a combined effect of clot-busting and prevention of further rebleeds. The key takeaway is that a combination of biologics may be used, and that intravitreal implantation or subretinal implantation may be potential ways to localize one, two, or more biologic therapies for enhanced effectiveness.

For glaucoma, first-line therapies are typically administered via use of daily eye drops, e.g. latanoprost (Xalatan, Pfizer). Glaucoma typically occurs when the drainage canals in the front of the eye (anterior) eye are slowly or quickly clogged or blocked, which raises eye pressure via liquid buildup and can damage the optic nerve. Once the optic nerve is damaged due to the pressure, vision loss occurs. Small molecules which increase uveoscleral fluid outflow, such as latanoprost, have been researched first as topical eye-drops (requires daily dosing, has been commercialized) and later in intravitreal implants (sustain its release up to ˜7 months, not commercialized).⁹ A takeaway is that there is potential for polymeric intraocular (for either anterior or posterior) implants, for treating either open-(90% of cases), closed-angle, or other types of glaucoma, delivering effective small molecule doses over a sustained period of time. ⁹ William White, et al., “In Vivo Pharmacokinetics of Injectable, Intravitreal, Sustained-Release Latanoprost Formulations,” Investigative Ophthalmology & Visual Science June 2013, Vol. 54, 5047. (meeting abstract, no doi)http://iovs.arvojournals.org/article.aspx?articleid=2149967

Biologics are not commonly used to treat glaucoma, though they have been considered for the disease. Biologics are quite challenging to preserve intraocularly for long periods of time. One example is Tissue Plasminogen Activator (tPA), which is a biologic shown to reduce eye pressure by: 1) treating the blockage or clogging of the drainage path of the eye via proteolytic degradation of fibrin^{10, 11} and 2) activation of other pro-enzymes leading to degradation of extracellular matrix components around the trabecular meshwork.¹⁰ tPA has a short half-life in the eye-less than 9 days.¹² Delivery methods of tPA into the eye have been suggested using topical administration, iontophoresis, engineered cell implantation, and slow release devices.¹³ One embodiment of our invention delivers tPA into the eye using the biodegrading sustained release implant, but also includes major differentiating and non-obvious design integrations for tPA to stabilize it and control its release (discussed in detail section below). Implantation of tPA using our DDS could be administered intracamerally or intravitreously. ¹⁰ Oscar A. Candia, et al., Tissue plasminogen activator reduces the elevated intraocular pressure induced by prednisolone in sheep, Experimental Eye Research, Volume 128, November 2014, Pages 114-116, ISSN 0014-4835, http://dx.doi.org/10.1016/j.exer.2014.10.004. (http://www.sciencedirect.com/science/article/pii/S0014483514002 681)¹¹H. Roger Lijnen, Desire Collen, 1 Mechanisms of physiological fibrinolysis, Bailliere's Clinical Haematology, Volume 8, Issue 2, 1995, Pages 277-290, ISSN 0950-3536, http://dx.doi.org/10.1016/S0950-3536(05)80268-9. (http://www.sciencedirect.com/science/article/pii/S0950353605802689)¹² Keller, K. E., Aga, M., Bradley, J. M., Kelley, M. J., & Acott, T. S. (2009). “Extracellular matrix turnover and outflow resistance. Experimental Eye Research,” 88(4), 676-682. DOI: 10.1016/j.exer.2008.11.023¹³ US2015/0366953, ¶94

Implants have been marketed for use in sustained release of intravitreally implanted drug delivery of small molecules. Small molecules may be innately more stable than biologics or other proteins. A biodegrading implant precedent, Ozurdex (dexamethasone implant) shows that intravitreal sustained release injection is possible to be commercialized. Ozurdex is a PLGA filament with steroid small molecules loaded on the surface; PLGA degrades under general hydrolysis and is bio-resorbed. Other non-biodegrading products releasing small molecules include Vitrasert, Retisert, and Iluvien are now commonly used. A common design between Vitrasert and Retisert is a cup with suture-tab. Iluvien uses a non-biodegrading tube with two semipermeable ends, delivering small molecules intravitreally for three years. These approaches do not integrate biologics, since these approaches may not appropriately sustain the lifetime of the protein in the hydrolytic and enzymatic environment of the intravitreal space.

Biologics-containing implants for ocular use have been investigated. There are limited precedents for delivering biologics for glaucoma intraocularly, either intravitreally (posterior) or in the intracamerally (anterior) in the eye.¹³ And while neovascularization in the posterior of the eye is typically treated intravitreously, there is no sustained release formulation available for the marketed biologic therapies.

Some advanced intraocular delivery concepts include combinations of sub-classes of the following administration methods: injection (conventional solution/suspension), vesicular, particulate, controlled release, or advanced. While each have drawbacks on their own, our invention combines the best attributes of each, to achieve sustained release. Our biologic vesicular encapsulation “stabilizer” design stabilizes the biologic against the microenvironmental pH. A further hydrogel environment envelops this vesicle: the hydrogel “core” is non-toxic and degrades with limited “burst” of the vesicles (controlled release). The “shell” of PLGA is a similar material which Ozurdex uses, but is designed in a different way, as a shell, to encapsulate the controlled release hydrogel. Each of the “Stabilizer-Core-Shell” components are designed to complement each other.

Several prior art disclosures provide erodible drug delivery devices for intraocular implantation. For example, WO2005/107727 provides an intraocular implant for the delivery of steroids in a biodegradable polymer matrix. No disclosure of biopharmaceuticals is provided, and the use of triblock polymers was not disclosed. In another example, WO2005/051234, discloses an injectable drug delivery device suitable for intraocular use. Its use with an array of small molecule drugs is disclosed. The use of this device with biopharmaceuticals is not provided, and the use of triblock polymers is not disclosed. In another example, U.S. Pat. No. 9,668,915 discloses implantable devices for treating ocular diseases, but does not provide triblock polymers, and does not discuss stabilizing protein therapeutic agents with a suitable in-situ buffer that maintains an appropriate pH to prevent degradation.

Thus, biologics are not easy to deliver intraocularly in a sustained release dosage form. While other ocular small molecule and biologic therapy administration methods exist, each administration method has significant drawbacks—ranging from inherent challenges of dosing efficiency, non-biodegradability, poor sustained release, to implicit issues with patient regimen persistence, economic cost, and usability. Intraocular implant precedents exist and a combination of techniques may improve the usefulness of the intraocular implant.

### SUMMARY OF THE INVENTION

In an embodiment, an ocular implant for the intraocular delivery of a therapeutic agent is provided. The implant may have a sustained-release core including a biodegradable gel medium, an active therapeutic agent, and a stabilizer. The implant may have a shell of biodegradable material covering the sustained-release medium. In an embodiment, the shell biodegradation half-life is longer than the release life of the core. The shell degradation half-life is the time required for the shell mass to reduce to half its initial value. The release life of the core is the time required for the quantity of the active therapeutic ingredient of the core to reduce to about 25% to about 0% of its initial value. In an embodiment, the implant is inserted into the vitreous body, anterior chamber, subretinal, or submacular area of an eye.

In an embodiment the implant dissolves after implantation into an eye, and is not removed.

The therapeutic agent may be a small-molecule drug or a protein-based biologic agent. In an embodiment, the drug may be selected from a small molecule substance such as:

tyrosine kinase inhibitors: nintedanib, Sunitinib;¹⁴

homoisoflavonoids: (cremastranone);¹⁵

anti-viral agents: ganciclovir, trifluridine, fluorometholone;¹⁶

antibiotics/aminoglycosides: neomycin, polymyxin, tobramycin, gentamicin, sulfacetamide;¹⁷

steroid anti-inflammatory agents: loteprednol, rimexolone, hydrocortisone, dexamethasone, methylprednisolone, prednisolone, flucocinolone, difluprednate, triamcinolone;¹⁸

prostaglandin analogs: unoprostone, latanoprost, bimatoprost, travoprost, tafluprost;^{19, 20}

miotics: echothiophate iodide, phospholine iodide, carbachol, acetylcholine;

beta blockers: levobunolol, betaxolol, carteolol, metipranolol, timolol;

alpha agonists: aprclonidine, brimonidine, lopidine;

carbonic anhydrase inhibitors: dorzolamide, brinzolamide, acetazolamide, methazolamide,

and mixtures thereof. ¹⁴ Mark B. Abelson and James McLaughlin, “New Therapies: Of Kinases and Cascades,” Review of Ophthalmology, May 5, 2013, https://www.reviewofophthalmology.com/article/new-therapies-of-kinases-and-cascades (no doi).¹⁵ Lee B and al., The first synthesis of antiangiogenic homoisoflavanone, cremastranone, Org Biomol Chem, 2014 Oct. 21; 12(39):7673-7, doi: 10.1039/c4ob01604a. Epub 2014 Aug. 28¹⁶ http://reference.medscape.com/dmgs/antivirals-ophthalmic¹⁷ Medikonda Radhika, et al., “Pharmacokinetics of intravitreal antibiotics in endophthalmitis,” J. Ophthalmic Inflammation and Infection 2014, 4:22 DOI: 10.1186/s12348-014-0022-z.¹⁸ Valentina Sarao et al., Intravitreal Steroids for the Treatment of Retinal Diseases, The Scientific World Journal, Volume 2014, Article ID 989501, http://dx.doi.org/10.1155/2014/989501.¹⁹ Jayaganesh V. Natarajan et al., Sustained Release of an Anti-Glaucoma Drug: Demonstration of Efficacy of a Liposomal Formulation in the Rabbit Eye, PLOS, Sep. 9, 2011, http://dx.doi.org/10.1371/journal.pone.0024513.²⁰ Justin A. Schweitzer and Mitch Ibach, Sustained-Release Drug Delivery: the Future of Glaucoma Treatment, Glaucoma Today, November/December 2016, http://glaucomatoday.com/2016/12/sustained-release-drug-deliver-the-future-of-glaucoma-treatment/ (no doi).

In an embodiment, the active agent may be a biologic agent. In an embodiment, the active agent is tissue plasminogen activator (tPA), a tPA variant, functional derivative, or homolog; a small molecule tPA agonist, an RNA molecule that causes tPA upregulation; an RNA molecule or other agent that down-regulates a negative regulator of tPA expression or activity, and a gene therapy vector, and mixtures thereof. In an embodiment, the active agent is an anti-VEGF (Vascular Endothelial Growth Factor) agent for treating age-related macular generation, diabetic retinopathy, or retinal vein occlusions, selected from a group consisting of:

Anti-angiogenics: ranibizumab or its biosimilars, bevacizumab or its biosimilars;²¹

VEGF inhibitors: aflibercept or its biosimilars, tumstatin-transferrins,²²

Inflammatory eye disease: etanercept, infliximab (ocular Becht's disease), adalimumab, other Tumor necrosis factor antagonists, Anti-interleukins, Interferon alpha,²³

siRNA (OPKO, Allergan, Quarkpham),²⁴

aptamers,²⁵

stem cells,²⁶ ²¹ The CATT Research Group, Ranibizumab and Bevacizumab for Neovascular Age-Related Macular Degeneration, N Engl J med 2011; 364: 1897-1908, DOI: 10.1056/NEIMoal102673²² Stewart M W, Aflibercept (VEGF-TRAP): the next anti-VEGF drug, Inflamm Allergy Drug Targets, 2011; 10(6): 497-508.²³ Chiara Posarelli et al., Biologic Agents in Inflammatory Eye Disease, J Ophthalmic Vis Res. 2011 October; 6(4):309-316, https://www.ncbi.nlm.nih.gov/pmc/articles/PMC3306110/.²⁴ A Guzman-Aranguez et al., Small-interfering RNAs (siRNAs) as a promising tool for ocular therapy, Br J Pharmacol. 2013 October; 170(4): 730-747, doi: 10.1111/bph.12330²⁵ Daniel W. Drolet et al., Fit for the Eye: Aptamers in Ocular Disorders, Nucleic Acid Ther, 2016 Jun. 1; 26(3): 127-146, doi: 10.1089/nat.2015.0573.²⁶ Ben Shaberman, ARVO 2016: What Does It Take to Develop a Stem-Cell Therapy for the Retina?, May 3, 2016, http://www.blindness.org/blog/index.php/arvo-2016-what-does-it-take-to-develop-a-stem-cell-therapy-for-the-retina/#more-4823

Neuroprotection: GABA/adenosine agonists, Inhibitors: K+/Cl− cotransporter inhibitors, PLC/PKC, NOS/Calpain inhibitors, L-type Ca2+ channel inhibitors, Anticonvulsants, antiarrhythmics against Na+ influx,²⁷ and mixtures thereof. ²⁷ N. N. Osborne et al., Optic nerve and neuroprotection strategies, Eye (2004) 18, 1075-1084. doi:10.1038/sj.eye.6701588

In an embodiment, the implant is a device having a core and a shell. The active therapeutic agent may be incorporated into the core. In an embodiment, the active therapeutic agent is incorporated in the shell, or a second active therapeutic agent is incorporated into the shell. In an embodiment, both the core and the shell are biodegradable and fully resorbable, so once implanted, the device need not be removed. In an embodiment, the shell has a biodegradable half-life that is longer than the half-life than the core. The half-life of a biodegradable polymer is the time required for a mass to reduce to half its initial value.

In an embodiment, the implant may have a generally cylindrical shape, in which a bioerodable core is encapsulated by a tubular shell. The implant device may have end caps of the shell material at one or both ends of the cylinder, or both ends may be open. The open ends expose the bioerodable core to bodily fluids at the site of the implant. As the core erodes, the active agent is released into the biological fluids around the implant, thereby providing highly localized delivery of the drug to the specific site where the implant is placed.

In an embodiment, the implant may have one or more perforations through the shell to expose additional surface area of the core to contact with bodily fluids.

In an embodiment, the implant may have other shapes besides a cylindrical shape. For example, the implant may be spherical.

### BRIEF DESCRIPTION OF THE DRAWINGS

FIG. 1A shows an embodiment of a cylindrical implant device showing one open end.

FIG. 1B shows an embodiment of a cylindrical implant device showing a closed end of the implant and optional perforations.

FIG. 2 is a spherical embodiment of the implant, showing optional perforations through the shell.

FIG. 3 shows a plot of the release profile of BSA-FITC from the core and shell of a model device according to Example 2.

FIG. 4 shows a plot of the release profile of BSA-FITC from the core only of the model device according to Example 3.

FIG. 5 shows the percentage of the cumulative release of Bovine Serum Albumin as a function of release in inventive devices of BSA loaded triblock copolymer hydrogel, according to Example 6.

FIG. 6 shows the percentage of the cumulative release of LYZ as a function of time in inventive devices of LYZ loaded triblock copolymer hydrogel, according to Example 8.

FIG. 7 shows the percentage of the cumulative release of LYZ as a function of time of an LYZ loaded triblock copolymer hydrogel, according to Example 10.

### DETAILED DESCRIPTION

### Structure of Sustained Drug Release System (DDS)

The general idea of the inventive implant device is that it provides a shell generally encapsulating a core, wherein an active therapeutic agent is part of the core material. In an embodiment, both the shell and the core are made of bioerodable (also termed biodegradable or resorbable) materials, so that once implanted, the device is resorbed into the body and need not be removed. In an embodiment, the core erodes more quickly than the shell. In an embodiment, there may be one or more perforations or other openings through the shell material that expose the core directly to bodily fluids at the implant site. In an embodiment, the shell includes the active therapeutic agent incorporated into the material of the shell. In an embodiment, a second active therapeutic agent is incorporated into the material of the shell. The rate of release of the active therapeutic agent is controlled by the balance of the rate of erosion of the core, the shell, and the openings (if any) through the shell. Using this device, extended release of an active agent is achieved, for example, as short as one-day, or as long as one year.

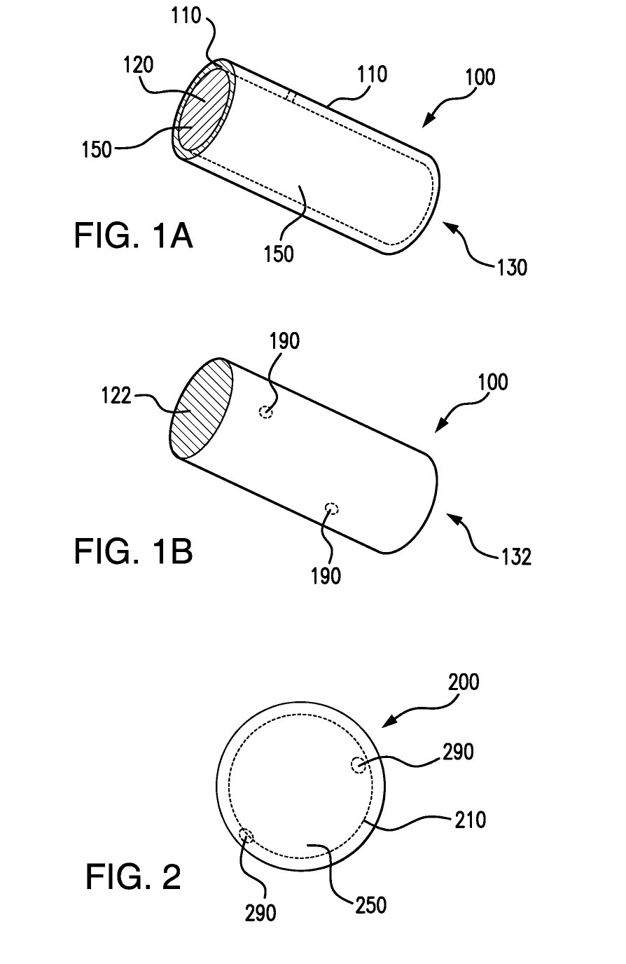

Referring to FIG. 1A, an embodiment of the DDS device 100 is shown having a hollow cylindrical shell 110, and bioerodable core 150, including a sustained-release medium comprising active therapeutic agents and component formulations for stabilizing the therapeutic agents. Also shown is an open end 120, exposing the core 150 to the local environment. The opposite end is 130 and may be open or sealed. FIG. 1B shows a sealed end 122. FIG. 1B also shows optional perforations 190, that increase the surface area of the core 150 to the bodily fluids of the local environment if present. The term “perforation,” means an opening in the shell that exposes the sustained-release biodegradable core medium to the physiological environment at the site of the implant. Thus, the sustained-release biodegradable core medium is in direct contact with bodily fluids at the location of a perforation in the shell after implantation. The embodiments illustrated in FIGS. 1A and 1B are referred to herein as “substantially cylindrical” in shape.

FIG. 2 shows an alternative embodiment 200 of the DDS which is spherical. In this embodiment, a spherical shell 210 may have perforations 290. This embodiment is shown with a spherical resorbable core 250 (shown in dashed lines). The embodiment illustrated in FIG. 2 is “substantially spherical” in shape. Other related geometric shapes are possible, for example, an elongated sphere.

### Shell

The shell may be made from biodegradable or bioresorbable polymers. In an embodiment, the shell can be a substantially hollow preformed cylindrical in shape, or “tube”. The shell is used to encapsulate the sustained release medium and to control the contact surface area for drug release from the medium. The shell could be a tiny tube or applied coating of outside diameter in the range of from about 0.1 mm to about 3 mm and length of from about 1 mm to about 10 mm.

If the outside diameter of the shell is greater than 3 mm, the dispensing needle used for injecting the DDS may be too large and cause undesirable side effects. If the outside diameter of the shell is less than 0.1 mm, the DDS may not be mechanically strong enough to sustain the force required to inject the DDS into the vitreous body. The inside diameter of the shell is pre-determined by the wall thickness of the shell. Generally, the wall thickness can range from about 0.01 mm to about 0.1 mm. If the wall thickness is too large, it may result in a small core that contains insufficient amount of sustained release medium. If the wall thickness is too small, the shell may not be mechanically strong enough to sustain the force required to push the DDS into the vitreous body. In addition, the mechanical strength of the biodegradable core can be important for the strength of the composite core-shell structure.

Optionally, the shell may have one or more perforations (delivery holes) for controlling the release rate which is, in the first order, proportional to the surface area contacting bodily fluids at the site of implantation. In an embodiment, the perforations can be cut into the shell using laser micro-machine method. In an embodiment, the holes can also be formed in casting or molding the shell. The diameter of laser micro-machined holes can range from 5 microns to 300 microns. The preferred range is from about 10 microns to about 100 microns.

In an embodiment of a substantially cylindrical shell, such as shown in FIG. 1B, the perforations may be along the cylinder walls as shown by perforations 190. In an embodiment, each end of the shell in a substantially cylindrical shell is open, that is, the shell has one or more perforations perpendicular to the longitudinal axis, for example perforations 120 in FIG. 1A. Alternatively, one end of the shell can be sealed (122 in FIG. 1B), leaving the other end open for release.

Optionally, the shell ends may be sealed with biodegradable semi-permeable membrane or mesh. Alternatively, the ends can be sealed with a biodegradable polymer. Such an end seal can provide a stable environment for a long shelf life. The end seal can also help attenuate any “burst” effect of drug release upon injection of the DDS into the vitreous body.

In an embodiment, the shell may include an active therapeutic agent incorporated therein. Any active therapeutic agent incorporated into the shell may comprise the same or a different therapeutic agent as is provided in the core comprising a biocompatible medium and an active therapeutic agent.

### Core Comprising Sustained Release Medium

Inside the shell as described above is filled with sustained release medium comprising a biocompatible medium and an active therapeutic agent. The core diameter is pre-determined by the mechanical strength of the biodegradable shell polymers, the outside diameter, and wall thickness of the shell, the sustained release biologics loading requirement, and the mechanical strength of the core. If the core medium is a hydrogel (a hydrophilic gel) which is generally a very soft composite, the core strength plays a negligible role in determining the core diameter and how the core-shell structure is made.

### Core and Shell Biodegradability Requirements

The shell plays a key role in providing a semi-closed, stable support for the biologics drugs. Generally, the biologics may require for example, a pH buffered environment. Otherwise its activity life is substantially shortened. For example, if it is desirable to have a four-month release life for the active therapeutic ingredient of the core, it may be desirable to select a biodegradable shell polymer of a degradation half-life longer than from about 4 to about 8 months. The release life of the core is the time required for the quantity of the active therapeutic agent of the core to reduce to from about 25% to about 0% of its initial value. The half-life of a biodegradable shell polymer is the time required for the mass of the shell to reduce to half its initial value. This may be a longer half-life than the biodegradable core, or it may be about the same, or it may be slightly shorter. The half-life of a biodegradable core is the time required for the mass of the core hydrogel to reduce to half its initial value. Ultimately the shell and core hydrogel all degrade, and degraded products are absorbed or flushed out the vitreous body (or other part of the eye where implanted) after they serve their functions. In an embodiment, the biodegradable implants of this invention are fully resorbed and need not be removed after insertion.

### Biodegradable Shell Materials

The exemplary biodegradable material of sufficient mechanical strength and degradation half-life is selected from a group of poly(lactide-co-glycolic acid) copolymer, poly(D-lactic acid), poly(L-lactic acid), poly(glycolic acid), and the mixtures of these biodegradable materials thereof. Generally, PLGA or poly(lactic-co-glycolic acid) is a copolymer which is used in a host of Food and Drug Administration (FDA) approved therapeutic devices, owing to its biodegradability and biocompatibility. The degradation products of this class of polymers are lactic acid and/or glycolic acid, which may change the pH value of the core environment thus cause the biologics to be ineffective. Thus, it is desirable to select a shell material that degrades after the core completes its drug release function. Poly(lactic acid) can have dexter (D) or laevus (L) chiral enantiomers. Generally, PLA or PGA homo-polymers are semi-crystalline containing crystalline and amorphous domains. The crystalline domains are impermeable to water thus much more resistant to hydrolytic degradation than the polymer chains in the amorphous domains. In one embodiment, the co-monomer lactide to glycolide ratio of poly(lactide-co-glycolide) can vary from 100 (polylactide homopolymer having D or L or DL mixed chirality) to zero (polyglycolide homopolymer). In one preferred embodiment, the co-monomer weight ratios are 100/0 lactide/glycolide, 94/6 lactide/glycolide, 85/15 lactide/glycolide, 75/25 lactide/glycolide, and 50/50 lactide/glycolide. The ranking of hydrolytic degradation half-life from long to short life is generally 100/0, 94/6, 85/15, 75/24 and 50/50.

In one embodiment, the shell polymer can be selected from a group consisting of poly(lactide-co-glycolide)-b-poly(ethylene glycol), poly(ε-caprolactone), poly(lactide-co-ε-caprolactone), poly(ε-caprolactone)-b-poly(ethylene glycol) diblock copolymers, poly(ε-caprolactone)-b-poly(ethylene glycol) triblock copolymers, poly(ε-caprolactone)-b-poly(ethylene glycol) multiblock copolymers, poly(ethylene glycol terephthalate)-b-poly(butylene terephthalate) multiblock copolymers, and mixtures thereof.

The biodegradable shell may also include a blend of both hydrophobic and hydrophilic polymers that accelerate or retard release of the active agents. Furthermore, it is believed that because hydrophilic polymer near the surface the shell has a greater compatibility with vitreous body fluid and a greater ability to take up water readily than the hydrophobic polymer, increasing the amount of hydrophilic polymer in the shell will result in a better control of release rate and degradation rate. However, because hydrophobic polymer is generally incompatible with hydrophilic polymer, the blend will result in macro-phase separation of the two polymers. One way of overcoming macro-phase separation is to form a shell with an A-B diblock copolymer blended with hydrophobic polymer, wherein A block is a hydrophilic polymer and B block is a hydrophobic polymer compatible with the hydrophobic polymer. Examples of A-B diblock copolymer, include but are not limited to, poly(ethylene glycol) (PEG)-b-poly(lactide-co-glycolide) (PLGA), molecular weight (MW) of PEG block from about 5,000 to about 1,000 Daltons, and MW of PLGA block from about 1,000 to about 5,000 Daltons. In forming a blend of PEG-b-PLGA diblock copolymer and PLGA, micro-phase separated PEG domains are present throughout the bulk of the shell. These hydrophilic microdomain can take up water and is compatible with vitreous fluid. The loading of A-B diblock copolymer in a shell can be from about 1% to about 40% by weight, or preferably from about 5% to about 25% by weight.

The shell of the invention comprises a second active therapeutic agent, which is different from a first biologic therapeutic agent in the core hydrogel, dispersed within a biodegradable polymer or a diblock copolymer/PLGA polymer blend. The shell compositions typically vary according to the preferred drug release profile, the active agent used, the condition being treated, and the medical history of the patient. Active agents that may be used include, but are not limited to, ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, and anti-angiogenic agents.

In one embodiment, the active agent is methotrexate. In another embodiment, the active agent is retinoic acid. In a preferred embodiment, the anti-inflammatory agent is a nonsteroidal anti-inflammatory agent. Nonsteroidal anti-inflammatory agents may be used include, but are not limited to, aspirin, diclofenac, flurbiprofen, ibuprofen, ketorolac, naproxen, and suprofen. In a more preferred embodiment, the anti-inflammatory

agent is a steroidal anti-inflammatory agent.

The steroidal anti-inflammatory agents that may be used in the shells include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

In one embodiment, cortisone, dexamethasone, fluocinolone, hydrocortisone, methylprednisolone, prednisolone, prednisone, and triamcinolone, and their derivatives, are preferred steroidal anti-inflammatory agents. In another preferred embodiment, the steroidal anti-inflammatory agent is dexamethasone. In another embodiment, the biodegradable shell comprises a combination of two or more steroidal anti-inflammatory agents.

The steroidal anti-inflammatory agent may constitute from about 10% to about 90% by weight of the shell. In one embodiment, the agent is from about 40% to about 80% by weight of the shell. In a preferred embodiment, the agent comprises about 60% by weight of the shell of the implant.

The use of extrusion methods allows for large-scale manufacture of shell and results in shell in the form of a tiny tube with a homogeneous dispersion of the drug within the shell polymer matrix. When using extrusion methods, the polymers and active agents that are chosen are stable at temperatures required for manufacturing, usually at least about 50° C. Extrusion methods use temperatures of about 25° C. to about 150° C., more preferably about 60° C. to about 130° C.

Different extrusion methods may yield shells with different characteristics, including but not limited to the homogeneity of the dispersion of the active agent within the polymer matrix. For example, using a piston extruder, a single screw extruder, and a twin-screw extruder will generally produce shells with progressively more homogeneous dispersion of the active agent. When using one extrusion method, extrusion parameters such as temperature, extrusion speed, die geometry, and die surface finish will have an effect on the release profile of the shells produced.

In one embodiment of producing shells by extrusion methods the drug and polymer are first mixed at room temperature and then heated to a temperature range of about 60° C. to about 150° c., more usually to about 130° C. for a period of about 0 to about 1 hour, more usually from about 0 to about 30 minutes, more usually still from about 5 minutes to about 15 minutes, and most usually for about 10 minutes. The shell tubes are then extruded at a temperature of between about 60° C. to about 130° C., preferably at a temperature of between about 75° C. and 110° C., and more preferably at a temperature of about 90° C.

In a preferred extrusion method, the powder blend of active agent and PLGA is added to a single or twin-screw extruder preset at a temperature of about 80° C. to about 130° C., and directly extruded as a tube with minimal residence time in the extruder. The extruded tube has then the loading dose of active agent appropriate to treat the medical condition of its intended use.

In a preferred embodiment, multiple extrusion method can be used to improve the dispersion of an active therapeutic agent in the shell matrix. In an example of double extrusion, the powder mixture comprising of shell polymer and active therapeutic agent is added to a single or twin-screw extruder fitted with a cylinder die to produce a continuous cylinder. The cylinder is then pelletized, and the hammer-milled pellet is further ground using a jet mill to produce powder. The jet-milled powder is then added to a single or twin extruder fitted with a hollow cylindrical die to produce shell comprising shell polymer and active therapeutic agent, of desirable size and wall thickness. In another embodiment, the twice extruded and jet-milled powder can be added to a single or twin extruder to produce a shell of suitable size and wall thickness.

In one embodiment, the use of dip coating or spray coating or inkjet coating methods also allows for large-scale manufacture of shell and results in shell in the form of a coating with a homogeneous dispersion of the drug within the shell polymer matrix. When using coating methods, the polymers and active agents that are chosen are soluble in a solvent required for manufacturing, usually dichloromethane, methanol, or acetone. Coating methods use temperatures of about 25° C. to about 60° C., depending the coating solvent used for manufacture. Coating is usually carried out at about room temperature, then heated to an elevated temperature to remove solvent, resulting in a solid coating on a core.

### Biodegradable Core Medium

Research has been conducted on many nanosystems and sub classifications which include liposomes, micelles, dendrimers, nanoparticles, and hydrogel approaches. This invention integrates major nanosystem classifications. There has been limited study on the combination of two or more such concepts, for example, hydrogels and nanomicelle/liposomes. Of note, researchers have proven that hydrogels can extend the release of bevacizumab (Avastin; a biologic) to 4 months (in rats using a light-activated suprachoroidal injection). Some liposome micellar hydrogels have been able to sustain release of small molecules (but not biologics) for nearly one year. There is limited precedent for simultaneous implementation of both hydrogels and nanomicelle/liposome designs. Additionally, this invention may include other polymeric biodegradable materials like PLGA, which have been investigated and used as a common biomaterial.

In an example, amphiphilic block copolymers (ABCs) were investigated for use as a sustained-release core medium for biologics drug delivery. The utility of ABCs for delivery of therapeutic agents results from their unique chemical composition, which is characterized by a hydrophilic block that is chemically tethered to a hydrophobic block. The hydrophilic block can be soluble in or swollen by water and is biocompatible with the biologics to be delivered while the hydrophobic block can serve as cohesive domain that fixes the core in its desirable shape and position. A water swollen hydrophilic domain could be thought of as a localized hydrogel domain because the domain size and shape can be controlled by the hydrophobic block. The relative chain length of hydrophilic to hydrophobic block can govern the amount of the drug the medium can hold in the biologics compatible domain and the drug release rate. Exemplary ABC's can be diblock (B1-b-B2), triblock (B1-b-B2-b-B1 or B2-b-B1-b-B2) or multi-block ([B1-b-B2]ₙ copolymers. B1 block is hydrophobic, B2 block is hydrophilic, and b stands for a chemical linkage between blocks. B1 or B2 block may comprise crosslinkable monomers such that polymer can be crosslinked to form a swelling resistant core. There are many biodegradable, biocompatible and FDA approved B1 or B2 block polymers. Examples of hydrophobic blocks approved by FDA are poly(lactic acid), poly(glycolic acid), or poly(lactic acid-co-glycolic acid), poly(ε-caprolactone), and copolymers of monomers of lactic acid, glycolic acid, caprolactone, and combination of these monomers thereof. Poly(ε-caprolactone) constituent is generally more resistant to hydrolytic degradation because of its longer hydrocarbon segment and it may form crystalline domains, and its hydrolytic degradation product is less acidic than the poly(lactide) or poly(glycolide) or poly(lactide-co-glycolide) copolymer. A frequently used hydrophilic block is polyethylene glycol or poly(ethylene oxide-co-propylene oxide) or poly(ethylene oxide)-b-poly(propylene oxide). The examples of hydrophilic block are not limited to those containing ethylene oxide monomers. Molecular weight of each block can range from about several hundred to about several hundred thousand Daltons. The preferred molecular weight of each block ranges from about several hundreds to about one hundred thousand Daltons.

In aqueous solution, polymeric micelles can be formed via the association of ABCs into nanoscopic core/shell structures at or above the critical micelle concentration. Upon micellization, the hydrophobic core regions serve as reservoirs for hydrophobic drugs, which may be loaded by chemical, physical, or electrostatic means, depending on the specific functionalities of the core-forming block and the solubilizate. Although the Pluronics®, composed of poly(ethylene oxide)-block-poly(propylene oxide)-block-poly(ethylene oxide), are the most widely studied ABC system, copolymers containing polyester hydrophobic blocks have also shown great promise in delivery applications. Because each ABC has unique advantages with respect to drug delivery, it may be possible to choose appropriate block copolymers for specific purposes, such as prolonging circulation time, introduction of targeting moieties, and modification of the drug-release profile. ABC's have been used for numerous pharmaceutical applications including drug solubilization/stabilization, alteration of the pharmacokinetic profile of encapsulated substances, and suppression of multidrug resistance. The solution properties of a suitable block copolymer are keys to a uniform mixture of the block copolymer medium and the biologic drug and stabilization components. This can be an important factor for manufacturing the core.

In solid state or concentrated solution, domain ordering results in the formation of a periodic distribution of B1 and B2, and many geometries have been observed. Below the transition temperature, however, the monomer segments will segregate and form regular, periodic structures. That is, the B1 and B2 segments of each copolymer chain will come together and display macroscopic order. Some of the common shapes that have been observed experimentally are lines (lamellar), hexagonal cylinders, and stacked balls (body centered cubic, BCC). Copolymers are important as a class of materials because they can be engineered to exhibit specific physical properties. Each geometry could potentially possess different physical characteristics, and thus the ability to readily switch between the phases could allow for materials with tunable properties. In a block copolymer, the hydrophilic phase can hold hydrophilic drugs and the hydrophobic phase can hydrophobic drugs. However, it is necessary to select a B1/B2 block ratio that is optimal for hold each combination of drug and stabilizer.

Thermal properties of a block copolymer solution are also critical for positioning the core loaded with a drug and stabilizer formulation in the shell. Certain classes of block copolymers form an aqueous solution of low viscosity at a temperature between 4° C. and room temperature while forming a hydrogel at mammalian body temperatures such as 37° C. The thermo-gelling or thermo-responsive properties of a block copolymer solution allow several possible ways of positioning the drug loaded core in the shell.

Thermo-gelling triblock copolymer B1-b-B2-b-B1 can be selected from a group of poly(lactide-co-glycolide)-b-poly(ethylene glycol)-b-poly(lactide-co-glycolide) [L/G=50/50 w/w], poly(lactide-co-glycolide)-b-poly(ethylene glycol)-b-poly(lactide-co-glycolide) [L/G=75/25 w/w], poly(caprolactone)-b-poly(ethylene glycol)-b-poly(caprolactone), poly(D,L-lactide)-b-poly(ethylene glycol)-b-poly(D,L-lactide)

B1-b-B2-b-B1 triblock copolymers, where the B1-blocks are a relatively hydrophobic poly(lactide-co-glycolide) and the B2-block is a relatively hydrophilic polyethylene glycol, have a hydrophobic content of about 50 to about 85% by weight and an overall block copolymer molecular weight of between about 3 kDa and 5 kDa can exhibit water solubility at low temperatures and undergo reversible thermal gelation at mammalian physiological body temperatures. At such high hydrophobic content, it is unexpected that such block copolymers would be water soluble. It is generally taught that any polymer having a hydrophobic content more than 50% by weight is substantially insoluble in water and can only be made appreciably soluble in aqueous systems, if at all, when a certain amount of an organic co-solvent has been added.

The biodegradable, hydrophobic B1-block segments can be poly(.alpha.-hydroxy acids) derived or selected from the group of poly(D,L-lactide-co-glycolide) and poly(L-lactide-co-glycolide), referred to collectively as poly(lactide-co-glycolide) or PLGA. If the average molecular weight of each of the B1-blocks in an B1-b-B2-b-B1 triblock copolymer is essentially the same, the average molecular weight (in Daltons) of each poly(lactide-co-glycolide) polymeric B1 block (e.g., PLGA.sub.1.5K) can be between about 1050 Da and about 1950 Da. For example, the average molecular weight of poly(lactide-co-glycolide) in the polymers may be from about 800 Da to about 1800 Da, from about 1000 Da to about 1700 Da, from about 1200 Da to about 1600 Da, or about 1400 Da to about 1500 Da. In certain embodiment, the average molecular weight may be about 800 Da, about 900 Da, about 1000 Da, about 1100, Da, about 1200 Da, about 1300 Da, about 1400 Da, about 1500 Da, about 1600 Da, about 1700 Da, or about 1800 Da, about 1900 Da, or ranges between any two of these values (including endpoints).

In one embodiment, the co-monomer lactide to glycolide ratio of poly(lactide-co-glycolide) can vary from 100 (polylactide homopolymer having D or L or DL mixed chirality) to zero (polyglycolide homopolymer). In one preferred embodiment, the co-monomer ratios are 100/0 lactide/glycolide, 85/15 lactide/glycolide, 75/25 lactide/glycolide, and 50/50 lactide/glycolide.

In one embodiment, the B1 block of B1-b-B2-b-B1 triblock copolymers can be endcap with crosslinkable monomers. In one preferred embodiment, the triblock copolymer is endcap with diacrylate. The triblock copolymer diacrylate can be UV cured with a photoinitiator such as Irgacure™ 2959. One example is poly(DL-lactide)-b-poly(ethylene glycol)-b-poly(DL-lactide) diacrylate endcap, wherein poly(DL-lactide) and poly(ethylene glycol) have molecular weights of 1700 and 1500, respectively. Thus this diacrylate terminated triblock copolymer does not only undergo thermogelling, but also can be further UV cured using Irgacure™ 2959. The resulting core can control the degree of osmotic swelling. The term “thermogel” means an aqueous polymer solution that undergoes sol-to-gel transition as the temperature increases. Thermogels that may be useful in this invention may be handled and administered as liquids, but form a gel deposit upon reaching body temperature.

In yet another embodiment, the B1 block of B1-b-B2-b-B1 triblock copolymers can be poly(lactide-co-caprolactone) [PLCL] or poly(caprolactone) [PCL]. In one preferred embodiment of this class of triblock copolymers, B1 PLCL or PCL block can have an average molecular weight of about 800 Da to about 2500 Da and B2 PEG block can have an average molecular weight of about 800 Da to about 2500 Da. Examples are poly(caprolactone)-b-poly(ethylene glycol)-b-poly(caprolactone), PCL-PEG-PCL (Mw˜1,000:1,000:1,000 Da), poly(lactide-co-caprolactone)-b-poly(ethylene glycol)-b-poly(lactide-co-caprolactone), PLCL-PEG-PLCL Mw˜1600-1500-1600 Da, 75/25 CL/LA, poly(lactide-co-caprolactone)-b-poly(ethylene glycol)-b-poly(lactide-co-caprolactone), PLCL-PEG-PLCL (˜1700-1500-1700 Da, 60/40 CL/LA). Triblock copolymers can be selected from a group of copolymers poly(glycolide-co-caprolactone)-b-poly(ethylene glycol)-b-poly(glycolide-co-caprolactone) of different glycolide to caprolactone ratios (PGCL-b-PEG-b-PGCL G to CL ratio from 100 to 0), and mixtures thereof.

ABC amphiphilic block copolymers in the form of B2-b-B1-b-B2 can be thermogelling wherein B2 is a hydrophilic polymer segment such as poly(ethylene glycol), and B1 is selected from a group of copolymers of two or more glycolide, lactide and caprolactone monomers. Examples are EG₁₂-(L₂₆G₇)-EG₁₂ 550-2320-550, EG₁₇-(L₂₆G₇)-EG₁₇ 750-2370-750, EG₁₂-(L₂₉G₈)-EG₁₂ 550-2600-550, EG₁₂-(L₃₁G₉)-EG₁₂ 550-2810-550, EG₁₂-(L₃₂G₉)-EG₁₂ 550-2910-550, EG₁₂-(L₃₁G₁₂)-EG₁₂ 550-2930-550, EG₁₂-CL₂₀-EG₁₂ (PEG-b-PCL-b-PEG) 550-2200-550, and mixtures thereof. EG, L, G, and CL stand for ethylene glycol, lactide, glycolide, and caprolactone monomer unit, respectively. The subscripted number stand for the number of monomer units in the block. The number stand for the molecular weight of each block.

Yet another embodiment of block copolymers exhibits gelling characteristics upon heating to 37° C. In one embodiment, the core polymer medium can be diblock copolymers B2-b-B1 wherein B2 is a hydrophilic block and B1 is a hydrophobic block. Examples are methoxy poly(ethylene glycol)-b-poly(caprolactone), mPEG-PCL (Mw˜750:2,400 Da), and methoxy poly(ethylene glycol)-b-poly(caprolactone-co-p-dioxanone), mPEG-b-PCDO, (MW˜750: 2400, 95/5 CL/DO).

As used herein, polyethylene glycol (PEG) may also be referred to as poly(ethylene oxide) (PEO) or poly(oxyethylene). The average molecular weight (in Daltons) of PEG in the polymers described herein may be about 700 Da to about 1300 Da. The average molecular weight of PEG in the polymers may be about 800 Da to about 2000 Da, about 900 Da to about 1500 Da, or about 1000 Da to about 1200 Da. In another embodiment, the average molecular weight may be about 700 Da, about 800 Da, about 900 Da, about 1000 Da, about 1100, Da, about 1200 Da, about 1300 Da, about 1400 Da, about 1500 Da, about 1600 Da, about 1700 Da, about 1800 Da, about 1900 Da, about 2000 Da, or ranges between any two of these values (including endpoints).

The thermogelling polymers investigated in this invention are soluble in water at ambient temperature and underwent sol-gel transitions with increasing temperature. The temperature driven sol-gel transition wherein the gelling behavior can be characterized by the critical gelling temperature (CGT) and critical gelling concentration (CGC). The sol-gel transition temperatures (T_{gel}) of triblock copolymer aqueous solutions with the indicated concentrations vary with the concentration of aqueous solution. The gel windows of aqueous polymer solution cover mammalian body temperatures range from about 36° C. to about 42° C., indicating that the thermogels are suitable for biomedical applications. Rheological properties as a function of temperature are often used to quantify the suitability of a thermogel polymer solution for ease of making and positioning core release medium inside the shell and sustained release applications. In this invention, the viscosity of the formulated aqueous core solution is adjusted to a range of from about 5 mPa-s to about 1,000 mPa-s at a temperature range from 5° C. to about T_{gel}. In addition, the storage modulus G′, loss modulus and tan(delta) of the invention are dynamic viscoelastic properties determined by temperature dispersion measurement method by sinusoidal oscillation method at an oscillation frequency of 6.28 rad/sec and 0.1% strain. For the measurement of these properties, AR550 measuring instrument produced by TA Instruments. may be used. In some detail, the core polymer medium solution is placed between a plate having a diameter of 60 mm and a 2-degree cone. The normal force is then adjusted to zero. Thereafter, the sample is given a sinusoidal oscillation at a frequency of 6.28 rad/sec. The measurement begins at 5° C. and then ends at 50° C. In this invention, the G′(5° C.) is adjusted to a range of from about 5 mPa to about 10 Pa. The ratio of G′(37° C.) to G′(5° C.) for the solution is adjusted from about 2 to about 20,000. A ratio range of from about 5 to 5,000 is preferred. A ratio ranges from about 10 to about 2,000 is further preferred.

The mixture of the biodegradable copolymer and drugs can be prepared as an aqueous solution of the copolymer below the gelation temperature to form a drug delivery system where the drugs can be either partially or completely dissolved, as described by the preparatory techniques described herein. When the drugs are partially dissolved, or, when the drugs are essentially insoluble, the drugs exist in a colloidal state such as a suspension or emulsion. This drug delivery system can then be administered intravitreally or inserted into a cavity such as by ocular administration to a patient whereupon it will undergo a reversible thermal gelation since body temperature will be above the gelation temperature.

The concentration at which the block copolymers are soluble at temperatures below the gelation temperature may be considered as the functional concentration. Block copolymer concentrations of as low as 3% and of up to about 50% by weight can be used and still be functional. However, concentrations in the range of about 5 to 40% are often suitable and concentrations in the range of about 10-30% by weight are particularly useful. To obtain a viable gel phase transition with the copolymer, a certain minimum concentration, e.g. about 3% by weight is required. At the lower functional concentration ranges the phase transition may result in the formation of a weak gel. At higher concentrations, a strong gel network is formed.

Another advantage to the composition described herein lies in the ability of the block copolymer to increase the chemical stability of many drug substances. Various mechanisms for degradation of drugs that lead to a drug's chemical instability can be inhibited when the drug is in the presence of the block copolymer. For example, biologics can be substantially stabilized in the aqueous polymer composition relative to certain aqueous solutions of the same drug in the presence of organic co-solvents. This stabilization effect can be achieved with many other hydrophobic drugs, provided that a stable thermogel can be prepared with the drug or drug combination in combination with the chosen amphiphilic polymer.

With respect to hydrogel or water-soluble polymer, crosslinked alginate, crosslinked hyaluronic acid, polyethylene oxide, crosslinked polyethylene glycol, polyethyleneimine, crosslinked polyethyleneimine or mixtures thereof can be used herein as non-thermogelling core medium or as an additive to the thermogelling block copolymers.

### Active Therapeutic Agent

In an embodiment, the active therapeutic agent is a biologic agent such as a therapeutic protein or nucleic acid, also termed a biopharmaceutical. In an embodiment, the biopharmaceutical is tissue plasminogen activator (tPA). In an embodiment, the biopharmaceutical is an anti-VEGF (Vascular Endothelial Growth Factor) protein. A therapeutic agent may be, for example, a protein, a glycoprotein, and antibody, an aptamer, or an oligonucleotide.

A tPA therapeutic agent may be selected from the group consisting of tPA, a tPA variant, functional derivative, or homolog; a small molecule tPA agonist, an RNA molecule that causes tPA upregulation; an RNA molecule or other agent that down-regulates a negative regulator of tPA expression or activity, and a gene therapy vector, and mixtures thereof.

Anti-VEGF (Vascular Endothelial Growth Factor) agents are used to treat age-related macular generation and diabetic macular degeneration. Anti-VEGF agents may be selected from bevacizumab, ranibizumab, aflibercept, Pegpleranib, biosimilar, and mixtures thereof.

Other protein therapeutic agents that may be effectively delivered with the inventive DDS include experimental agents such as Ocugen OCU100, OCU200 biologic, and Graybug GB-102.

In an embodiment, the therapeutic agent may be a small-molecule drug defined as a chemically synthesized product. In an embodiment, the drug may be selected from a small molecule substance such as:

tyrosine kinase inhibitors, for example nintedanib, Sunitinib;

homoisoflavonoids for example cremastranone;

anti-viral agents, for example ganciclovir, trifluridine, fluorometholone;

antibiotics, for example aminoglycosides, neomycin, polymyxin, tobramycin, gentamicin, sulfacetamide:

steroid anti-inflammatory agents, for example loteprednol, rimexolone, hydrocortisone, dexamethasone, methylprednisolone, prednisolone, flucocinolone, difluprednate, triamcinolone;

prostaglandin analogs, for example unoprostone, latanoprost, bimatoprost, travoprost, tafluprost; miotics, for example echothiophate iodide, phospholine iodide, carbachol, acetylcholine;

beta blockers for example levobunolol, betaxolol, carteolol, metipranolol, timolol;

alpha agonists, for example aprclonidine, brimonidine, lopidine;

carbonic anhydrase inhibitors, for example dorzolamide, brinzolamide, acetazolamide, methazolamide,

Mixtures of any of these products are also within the scope of this invention.

### Therapeutic Agent Stabilizer

Because of the extended release life of the active therapeutic agents, as a general proposition, a stabilizer is a desirable component in the implants as described herein. In an embodiment, the stabilizers are excipients that prevent or retard the chemical degradation of an active therapeutic agent after implantation. This may be necessary in some cases to ensure that the active therapeutic agent is available after a period of time in the implant. In various embodiments, the stabilizer may be a pH buffer solution, an albumin, a liposome, a PEGylated liposome, or a poly(vinyl pyrrolidone) material

For example, human tissue Plasminogen Activator (tPA) is a glycoprotein of about 70,000 Daltons and 527 amino acids. tPA is not highly soluble in aqueous solution. Its solubility in water at pH=7.0 is about 0.1 mg/ml. tPA is commonly solubilized with typical solubilizing agents. These include chaotropic denaturing agents such urea and thiocyanate, surfactants such as detergents, carbohydrates such as mannitol, and proteins such as albumin and gelatin. Although these solubilizing agents improve the water solubility of tPA, resulting tPA solutions tend to be less stable, and are not being recognized as being acceptable for certain administration, or must be employed in acceptably large amounts. It has been discovered that tPA is highly soluble when buffered in an environment at pH=3.5-4.5. For example, at pH 4.0 (0.1M ammonium acetate), the solubility of tPA is greater than about 15 mg/ml; at pH 5.0, the solubility of tPA is approximately 3 mg/ml. As stated previously, the solubility of tPA in water at pH=7.0 is about 0.1 mg/ml, while in the pH range from about 3.5 to about 5.5, tPA is highly stable. Such an enhanced stability is apparent at low as well as high concentrations of tPA. The core comprising tPA can be buffered with one or more of adipic acid, glutamic acid, citric acid, succinic acid, lactic acid, tartaric acid, and a salt thereof. In this invention, the shell of the drug delivery system can serve as outer wall encapsulating an environment for solubilizing and stabilizing tPA. A high temperature can be used to expedite the loss of tPA activity. Thus, in manufacturing a drug delivery system containing tPA, a temperature higher than about 45° C. should be avoided.

One advantage of the drug delivery system in this invention is the selection of the shell polymer and the core medium polymer. A shell polymer having a long biodegradation half-life will produce much less degradation products that can alter the pH environment in the core. Although the core medium polymer is biodegradable, a preferred polymer is selected such that the acidity of its degradation products is substantially minimized.

Although tPA can be stabilized and maintain its activity inside the drug delivery system of this invention, as soon as it is released into the vitreous body, it again encounters a harsh environment of pH=7.4 wherein tPA activity is decreased to zero in about two to six days. It is thought that tPA nano-encapsulation using liposomes can provide a longer half-life in the vitreous body environment. Liposomes are vesicular drug delivery systems that consist of lipid bilayer arrays. Each lipid bilayer consists of two parts hydrophilic and hydrophobic. The hydrophilic parts are directed toward aqueous phase, while the hydrophobic parts are directed toward each other. This structure gives liposomes the opportunity to encapsulate both water-soluble and water-insoluble bioactive materials. Liposomes are highly biocompatible with low immunogenicity, and they are also characterized by their efficient encapsulation of drugs. Their nano-encapsulation efficiency is often questionable because liposome molecules can be solubilized in certain environment. In an embodiment, a compatible protein such as albumin, gelatin, or a compatible polymer such as poly(vinyl pyrrolidone) (Plasdone C-30), poly(ethylene glycol) (e.g. molecular weight 4,000 to 40,000), Brij 700, polysorbate 80, or Tween 80 can shield a protein such as tPA from direct contact to pH=7.4 solution.

In an embodiment, an amino acid such as L-arginine can protect tPA from degradation and aggregation by binding to the surface hydrophobic regions of the protein. This effectively reduces the chance of intermolecular interactions among tPA molecules.

In another embodiment, a protein such as tPA is protected from degradation by nanoencapsulating the protein with a PEGylated liposome. This involves chemical modifications of the surface of the liposomes with substances such as poly(ethylene glycol) in a process called PEGylation. Poly(ethylene glycol) has several advantages, including high water solubility and low cytotoxicity. In addition, the PEGylated liposomes or vesicles demonstrate high stability due to steric repulsion which prevents the fusion and disruption of the vesicles. Moreover, the liposomal modification with PEG widens the applicability of liposomes by enhancing its circulation time in addition to its targeting capabilities. For instance, the PEGylated liposomes prolong the circulation of tPA. Encapsulation of tPA into conventional liposomes and PEGylated liposomes prolong the circulation time by about 16-fold to about 21-fold.

These stabilizing strategies can be generally applicable to many biologic protein drugs.

### Preparation of Core Comprising Medium, Drug and Stabilizer

A new procedure was developed that enables the preparation of a stable thermogel core. The core will contain active therapeutic agents, stabilizer, and a polymer matrix medium. First, prepare the combination of active therapeutic agent and stabilizer by dissolving the active in a suitable solvent system. Heating the active therapeutic agent and the solvent system to about 40-50° C. can aid in dissolving the active therapeutic agent. Next, the core medium polymer matrix is prepared by dissolving a B1-b-B2-b-B1 triblock copolymer in cold water. The aqueous polymer solution can be incubated at refrigeration temperature (e.g., about 4° C.) for a period sufficient to provide a solution of the polymer. For example, PLCL-PEO-PLCL triblock copolymer can be dissolved in water at about 4° C. The next step is to rapidly incorporate the polymer solution to the drug solvent solution. The mixture can be vortexed and warmed to 40-50° C. briefly to prevent phase separation. This mixture can then be rapidly frozen with ethanol/dry ice (−72° C.) and lyophilized to provide a cake or powder of the dehydrated formulation. Cold water (˜4° C.) can be added to the lyophilized sample to provide a clear solution of the drug delivery core comprising medium triblock copolymer, biologics protein drug, and stabilizer. The rehydrated solution can optionally be incubated at about 4° C. for 30 minutes prior to filtering the solution with a regenerated cellulose filter to remove materials unincorporated in the thermogel. The thermogel can then be diluted with cold solvent and the content of drugs incorporated can be quantified using applicable instrument analysis.

The biologic active therapeutic agent may constitute from about 2% to about 80% by total solids weight of the “core”. In one embodiment, the agent is from about 2% to about 60% by total solids weight of the core. In a preferred embodiment, the agent comprises from about 2% to about 45% by weight of the core of the implant.

### Preparation of Core-Shell Drug Delivery System

The stable thermogel “core” solution, comprising the active therapeutic, stabilizer, and polymer matrix, is encapsulated by a shell. This combination is thereby called a “core-shell”. A long core-shell tube filled, or “filament”, can be cut into segments of a desired length. The core will be either substantially dried, or if the filling is still hydrated, both ends of a cut filament can be sealed with removable plug to prevent the loss of core water from both ends of the shell tube segment.

The core solution gels at 37° C. In one embodiment, water can be removed from the core to achieve as much as from about 40% to about 90% solids (from about 60% to about 10% water by weight). For certain biologic therapeutic agents, the low water content core hydrogel is preferred because the core loses a substantial amount of water when exposed to phosphate buffer solution (pH=7.4). The low water content core is also loaded with a higher amount of active therapeutic agent per unit volume of the DDS than the high water-content core.

One way to incorporate the core into a preformed shell tube is: if the viscosity of the core solution is sufficiently low, the core solution can be vacuumed into a preformed shell tube, by connecting one end of the shell tube segment to a vacuum source, and dipping the other end of the shell tube into the core solution. The shell tube is filled with the core solution as soon as the vacuum is initiated from one end. This method may utilize a higher water content, and a drying phase may be used to pack more solids within the shell.

Another way to incorporate the core into a preformed shell tube is: If the viscosity of the core solution is medium to high, an injection method can be applied. For example, a nozzle is fitted to the preformed shell tube. A reservoir of core solution feeds the nozzle. To inject the core solution through the nozzle into the preformed shell tube, apply a high pressure to push the viscous core solution into the shell tube.

Yet another method is to form them together by co-extrusion into a filament. A continuous jet-stream of shell surrounding a continuous jet-stream of the core solution will create a “filament” of core insulated by the shell.

A final method to build the core-shell is using a dried core cylindrical filament and then applying a shell coating to it. One coating approach is to draw the dry filament through a sufficiently concentrated coating solution (hot acetone solution at about 45° C.) comprising the shell polymer, followed by drying the wet coated layer. A second coating approach is to dip the dried biologic containing core into a hot concentrated shell polymer solution, followed by removal of the solvent from the wet coating shell layer. A third coating approach is to use an injection layer printing process to apply a shell layer onto a cylindrical filament.

The implants as described herein provide a controlled release of the active therapeutic agent over a period of days, weeks, or months. In an embodiment, the release life of the active therapeutic agent in the implants as described herein is a period of time ranging from one day to one year. For example, the release life may be 7 days, 14 days, 21 days, 28 days, 6 weeks, 8 weeks, 10 weeks, 12 weeks, 16 weeks, 20 weeks, 24 weeks, 9 months, or 12 months.

The release rate of the therapeutic agents from the sustained release DDS formulation is controlled primarily by the core formulation, and its thermal and rheological properties at mammalian body temperature. Thus, it is critical to select an appropriate core hydrogel medium with the gel properties and biodegradation properties suitable for a predetermined long-term biologic protein release profile.

Other factors that affect the release rate include the chemical composition of the shell, and the nature and size of perforations in the shell that expose the core material to bodily fluids.

### Intravitreal Implantation of Sustained Release DDS

An exemplary sustained release DDS, a tiny cylinder of 0.5 mm OD and 8 mm long, is prepared. The cylindrical DDS is then injected into the central vitreous humor under topical anesthesia. For the injection, a disposable syringe with a 12.7 mm 21 gauge was used to penetrate the globe to the full length of the needle at a point approximately 4 mm posterior to the limbus. The angle of penetration was such that the tip of the needle was centrally located within the vitreous humor. After the DDS is injected into vitreous humor, the DDS will settle down to the inferior area of the vitreous humor.

### Intracameral Implantation of Sustained Release DDS

Investigation results showed a single injection of tPA into the anterior chamber (AC) also reduced intraocular pressure (TOP). This would be important as an eventual treatment Animal experiments demonstrated that intracameral injection of tPA had a dose dependent effect. A low tPA/arginine dose that was injected in a volume of 50 μL (10 ng tPA dose) into the anterior chamber (AC), there were no signs of inflammation or toxicity. This data suggested that a smaller implant in the AC is needed than that in the vitreous humor. An exemplary implant of 0.2 mm OD can be injected intracamerally using 24-gauge to 27-gauge needle.

### Utility of the Invention

The ocular implants of this invention may be useful as drug delivery platforms that can deliver a therapeutic agent, in particular a biologic therapeutic agent (biopharmaceuticals), to an interior location of a mammalian eye that cannot normally be accessed by externally applied drugs, such as eye-drops or other externally applied therapeutic agents. Similarly, the interior chambers of the eye are not vascularized, so conventional drugs in the blood, orally ingested or injected, are not bioavailable to the internal anatomy of the eye, where many ocular diseases are localized. The eye locations where the ocular implants of this invention may be implanted include the vitreous body, anterior chamber, subretinal, or submacular parts of an eye. In particular, the ocular implants of this invention may be useful as drug delivery platforms for the controlled released of a therapeutic agent over a period of time ranging from one day to one year.

The ocular implants of this invention may be useful for any mammalian eye, in particular human eyes, but also eyes in non-human animals (veterinary use). For example, the implants may be used in companion animals, such as dogs and cats, and in livestock, such as pigs, goats, camels, llamas, cattle, and horses.

Diseases that may be treatable with biologic therapeutic agents delivered intraocularly with this invention include
Neovascularization disorders in the back of the eye, such as age-related macular degeneration, diabetic retinopathy, retinal vein occlusions, retinal artery occlusions.
Glaucoma-related ocular disease including open, closed-angle, or other forms.
Cancers (neoplasms) internally in the eye.

### EXAMPLES

### Example 1. Preparation of BSA-FITC Loaded Triblock Copolymer Solution

B1-b-B2-b-B1 triblock copolymer Poly(D,L-lactide)-b-Poly(ethylene glycol)-b-Poly(D,L-lactide) PDDLA-PEG-PDLLA triblock copolymer (1,700:1,500:1,700)

| | Number | Weight | |
|---|---|---|---|
| | Average | Average | Polydispersity |
| | Molecular | Molecular | Index |
| Polymer | Weight, Mn | Weight, Mw | PDI |
| PDLLA-b-PEG-b-PDLLA | 5,350 | 6,800 | 1.27 |
| PEG 1,500 Initiator | 1,485 | — | — |

Materials
The B1-b-B2-b-B1 triblock polymer was ordered from PolySciTech, Catalog Number: AK100
Protein: Bovine Serum Albumin-fluorescein isothiocyanate conjugate (BSA-FITC) was obtained from Protein Mods., product code BSF product lot 263BSF2, molecular weight ˜66 KDa, 100 mg/ml in H₂O. BSA modification level 0.9 luorescein/molecule.
Phosphate buffered solution (1M PBS, pH=7.4) was purchased from Sigma-Aldirch.
Acetone, reagent grade, was purchased from Fisher Scientific.

Procedure: In a 3 ml vial, 0.0966 g of B1-b-B2-b-B1 triblock copolymer was dissolved in 0.3807 g acetone, and 0.2288 g deionized distill water was added, and the vial was gently mixed for at least 30 minutes until a clear solution formed. The acetone co-solvent was evaporated fully at room temperature to give a final clear polymer aqueous solution. The polymer solution was 29.7% triblock polymer by weight. The polymer solution may be referred to as a “sol,” which gels upon heating to mammalian body temperature. A sol can mix with a biologic drug to form a solution which still maintains the gelling property.

To the above polymer solution, 0.0446 g of BSA-FITC solution was added. The BSA-FITC/polymer solution was thoroughly mixed. The protein loading was 4.41% by weight based on total weight of solids.

### Example 2. Preparation of Model Core-Shell DDS

To model a DDS shell, a 20 AWG miniature polyimide tube was used with an outside diameter of 962 microns, a wall thickness of 76.2 microns, and an inside diameter of 810 microns. The inside cross section area was 0.515 mm² The polyimide tube was not biodegradable, so in this case, the aim is to test the release capability of core through a fixed area.

The polymer solution of Example 1 loaded with BSA-FITC was injected into a ˜3″ long segment of the polyimide tube. The tube was incubated at 37° C. for 15 minutes, and the BSA-FITC loaded polymer solution formed a hydrogel. The tube was cut into about 9 mm-long sections. Each 9 mm tube section had an estimated 5.92 mg of the hydrogel is loaded inside the tube. The sections of polyimide tube containing BSA loaded triblock copolymer hydrogel were further incubated at 37° C. for about 15 minutes. Both ends of each tube segment were sealed with wax to prevent any loss of water before the protein release experiment started.

### Example 3 Preparation of BSA-FITC Loaded Hydrogel Core

The triblock copolymer and BSA-FITC solution in Example 1 was also used a comparative hydrogel. It is difficult to obtain an accurate measurement for the total surface area of the sample because of the curvature in the hydrogel surface, but based on the inside diameter on the vial, the initial release surface area is at least 126.7 mm², and the release surface area was about constant for the first month of release and shrank gradually. The composition of the hydrogel sol consists of 95.3 mg of triblock copolymer, 4.40 mg of BSA-FITC and water. The total solids concentration was 29.7% by weight. After the sol was placed in a glass vial and incubated at 37° C. for 15 minutes, it formed a gel. To this vial, 2 ml of 1M phosphate buffered solution (PBS) pH=7.4 already incubated at 37° C., was added to the gel and the PBS and the gel clearly remained in two phases at the start and over the course of the in vitro experiment. The time was recorded as zero after the hydrogel was immersed in the PBS.

### Example 4. In Vitro BSA-FITC Release Study of Core and Model DDS

The implant of two devices of BSA loaded triblock copolymer hydrogel from Example 2 was placed in a 3 ml screw cap vial with 2 ml PBS solution (pH=7.4) at 37° C. The vial was placed in a dry bath incubator at 37° C. The vials were incubated at 37° C. during the entire period of the study. Assay procedure: an aliquot of about 25% (˜0.5 ml) of the solution of the released BSA-FITC was removed for analysis, and then about the same amount of fresh PBS solution (already equilibrated at 37° C.) was added to the vial and the incubation resumed. Removal and assay of aliquots was conducted according to a pre-determined schedule.

Measurement procedure: BSA-FITC calibration solutions were made up with known BSA-FITC concentration. FluoroMax-4 from Horiba was used for measuring the peak fluorescence intensity in 510-550 nm spectral range. A calibration curve was generated by plotting peak intensity as a function of BSA-FITC concentration. Thus, by measuring the peak intensity of the assay solution, the BSA-FITC concentration in the aliquots is determined according to the calibration curve.

A mass balance was performed to find the cumulative amount of BSA-FITC solute:

Mᵢ=CᵢV+ΣCᵢ₋₁Vₛ

where Cᵢ is the concentration of solute in the release solution at time i, V is the total volume of the release solution (˜2 ml) and Vᵢ is the sample volume (˜0.5 ml). All florescence intensity measurements were performed at room temperature.

### Results

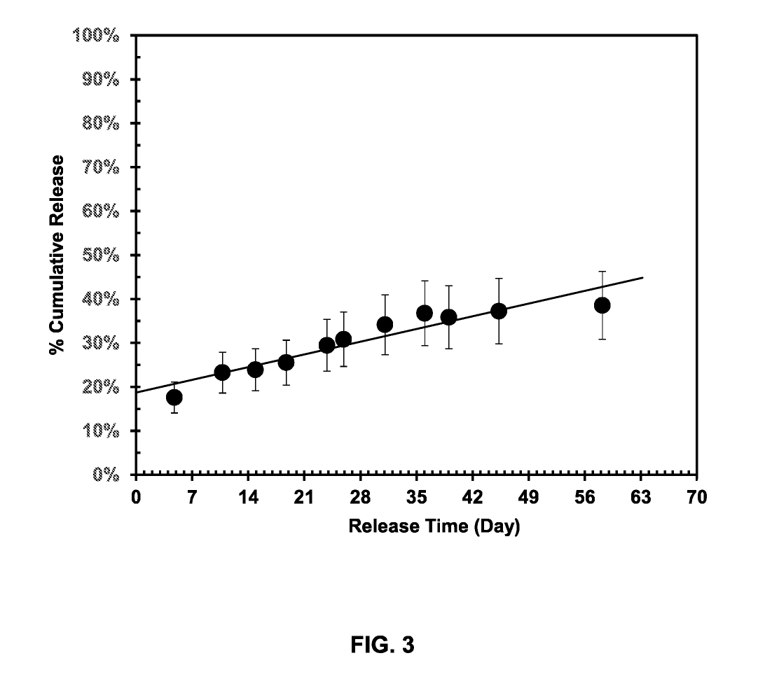

FIG. 3 shows the release profile from the model core-shell DDS (Example 2). The profile shows an initial burst release of BSA-FITC, which is not always observed in other similar prototypes. This initial burst amount of protein may be due to a small amount of hydrogel leakage resulting from the imperfect cutting of the tubing. Excluding the first burst data point, the release profile can be a good fit by a linear line with the R-square value of 0.91. The average payload calculated from the linear portion of the release profile is about 0.75 μg/day.

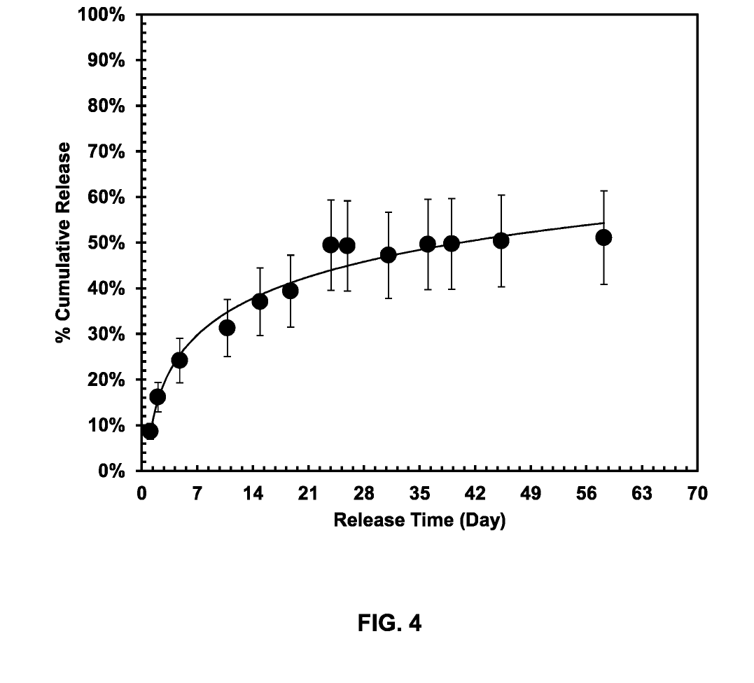

FIG. 4 shows the release profile of the core hydrogel alone (i.e., without the shell, Example 3). The profile shows an initial burst amount of BSA-FITC, which was always observed from other similar experiments. It is possible that this initial burst amount is attributed to the residual hydrogel sticking to the wall of the vial. The surface area could be varying during the 60 day release test. The solid line in FIG. 4 is simply a smooth line drawn through the data points.

### Example 5. BSA Loaded Triblock Copolymer Solution and DDS Device

Deionized distilled water, 0.2764 g, was added to AK100 B1-b-B2-b-B1 triblock copolymer, 0.0909 g, in a 3 ml vial. The vial was gently mixed for 2-3 days to form a clear solution. The polymer solution consisted of 24.8% by weight of the polymer. To the polymer solution, 0.0165 g of bovine serum albumin (BSA, obtained from Protein Mods) was added, and mixed thoroughly. The protein loading was 15.4% by weight based on total weight of solids. The % solids in the solution is 28.0%.

This polymer solution loaded with BSA was injected into an ˜5″ long PLGA biodegradable tube (0.508 mm OD and 0.356 mm ID, obtained from Biogeneral). The tube was incubated at 37° C. for 15 minutes, and the BSA loaded polymer solution formed a hydrogel. The tube was cut into about 5 mm sections. For each 5 mm tube section, there was an estimated 0.571 mg of the hydrogel with 0.0246 mg of BSA loaded inside each tube section. The sections were further incubated at 37° C. for 15 minutes. Both ends were sealed with wax to prevent any loss of water before the protein release experiment started.

### Example 6. In Vitro BSA Release Study of the Biodegradable DDS

The devices of BSA loaded triblock copolymer hydrogel in Example 5 were each placed in a 3 ml screw cap vial with 2 ml 1M PBS solution (pH=7.4) at 37° C. The vial was placed in a dry bath incubator at 37° C. Assay procedure: an aliquot of about 25% (˜0.5 ml) of the solution of the released BSA was removed for analysis, and then about the same amount of fresh PBS solution (already equilibrated at 37° C.) was added to the vial and the incubation resumed. Removal and assay of aliquots was conducted according to a pre-determined schedule.

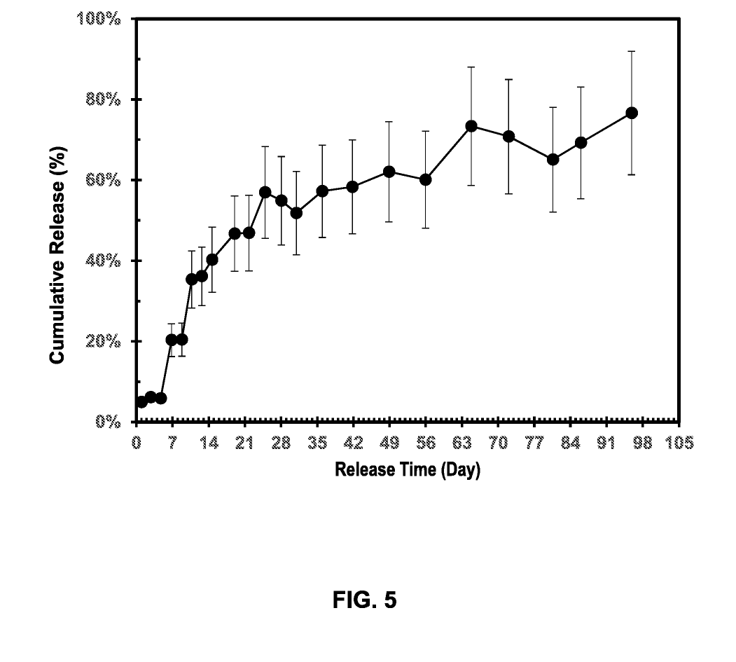

A Qubit Protein Quantification Device was used to determine the BSA concentration. The cumulative release percentage was determined according to the mass balance equation described in Example 4. FIG. 5 shows the % cumulative release as a function of release time.

### Example 7. Preparation LYZ Loaded Triblock Copolymer Solution and DDS

Deionized distilled water, 0.2573 g, was added to 0.0854 g of AK100 B1-b-B2-b-B1 triblock copolymer in a 3 ml vial. The vial was gently mixed for 2-3 days to form a clear solution. The polymer solution consisted of 24.9% by weight of the polymer. To the polymer solution, 0.0222 g of lysozyme (LYZ, molecular weight 14.4 KDa, from Sigma Aldrich) was added and thoroughly mixed. The protein loading was 20.7% by weight based on total weight of solids. The percentage of solids in the solution was 29.5%.

The triblock copolymer solution loaded with LYZ was injected into an ˜5″ long PLGA biodegradable tube (0.508 mm OD and 0.356 mm ID, obtained from Biogeneral). The tube was incubated at 37° C. for 15 minutes, and the LYZ loaded polymer solution formed a hydrogel. The tube was cut into about 5 mm sections. For each 5 mm tube section, there was an estimated 0.571 mg of the hydrogel with 0.0347 mg of LYZ loaded inside each 5 mm tube. The sections were further incubated at 37° C. for 15 minutes. Both ends were sealed with wax to prevent any loss of water before the protein release experiment started.

### Example 8. In Vitro Release Study of Biodegradable LYZ Loaded DDS

The LYZ loaded triblock copolymer hydrogel devices of Example 7 were each was placed in a 3 ml screw cap vial with 2 ml 1M PBS solution (pH=7.4) at 37° C. The vial was placed in a dry bath incubator at 37° C. Assay procedure: an aliquot of about 25% (˜0.5 ml) of the solution of the released LYZ was removed for analysis, and then about the same amount of fresh PBS solution (already equilibrated at 37° C.) was added to the vial and the incubation resumed. Removal and assay of aliquots was conducted according to a pre-determined schedule.

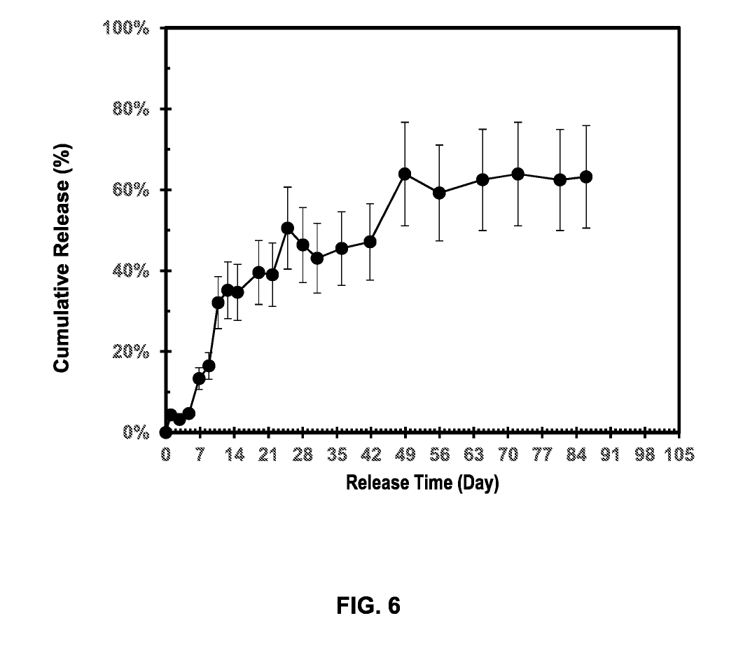

A Qubit Protein Quantification Device was used to determine the LYZ concentration. The cumulative release percentage was determined according to the mass balance equation described in Example 4. FIG. 6 shows the % cumulative release as a function of release time.

### Example 9. Preparation LYZ Loaded Triblock Copolymer Solution

Deionized distilled water, 0.2158 g, was added to 0.0603 g of AK100 B1-b-B2-b-B1 triblock copolymer in a 3 ml vial. The vial was gently mixed for 2-3 days to form a clear solution. The polymer solution consisted of 21.8% by weight of the polymer. To the polymer solution, 0.0302 g of lysozyme (LYZ, obtained from Sigma Aldrich). was added and thoroughly mixed. The protein loading was 33.4% by weight based on total weight of solids. The % solids in the solution is 29.5%.

### Example 10. In Vitro Lysozyme Loaded Hydrogel Release Study

The LYZ loaded triblock copolymer solution of Example 9 was placed in a 3 ml screw cap vial with 2 ml 1M PBS solution (pH=7.4) at 37° C. The vial was placed in a dry bath incubator at 37° C. The PBS and the gel were observed to remain in two phases at the start and over the course of the experiment. The vial was placed in a dry bath incubator at 37° C. Assay procedure: an aliquot of about 25% (˜0.5 ml) of the solution of the released LYZ was removed for analysis, and then about the same amount of fresh PBS solution (already equilibrated at 37° C.) was added to the vial and the incubation resumed. Removal and assay of aliquots was conducted according to a pre-determined schedule.

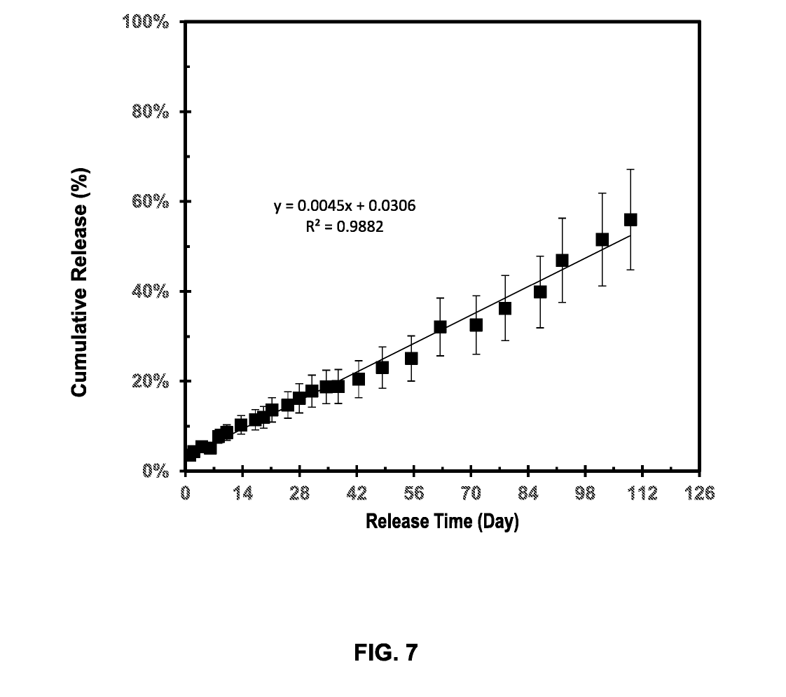

A Qubit Protein Quantification Device was used to determine the LYZ concentration. The % cumulative release was determined according to the mass balance equation described in Example 4. FIG. 7 shows the % cumulative release as a function of release time. It is apparent that this release profile is highly linear until about 55% of the LYZ was released at 110 days.

### Example 11 r-tPA and Stabilizer Loaded Triblock Copolymer Solution

Tissue plasminogen activator recombinant, r-tPA (an active therapeutic biologic for treating elevated steroid-induced intraocular pressure), molecular weight 59K Da) is obtained from Creative Enzyme (catalog # NATE-0920). Human serum albumin, HSA is purchased from Sigma-Aldrich (SKU A3782). 1×PBS tablets are obtained from Millipore Sigma (SKU P4417). 1×PBS pH 7.4 solution is prepared by dissolving one 1×PBS tablet in 200 ml of deionized distill water. Dissolve 1.8 mg of r-tPA and 1.2 mg of HSA in 5 ml of 1×PBS pH 7.4 solution. The r-tPA and HSA stabilizer solution is lyophilized to obtain about 3.0 mg of HSA stabilized r-tPA complex powder.

Add 0.0854 g of AK100 B1-b-B2-b-B1 triblock copolymer with 0.2573 g of deionized distill water. Gently mix the mixture to form a clear solution in a 3 ml vial. The final clear polymer aqueous solution was formed after 2-3 days. The polymer solution comprises 24.9% by weight of the polymer. To 0.0343 g of the polymer solution, add 0.00222 g of the HSA stabilized r-tPA powder. Mix the HSA stabilized r-tPA powder with the polymer solution thoroughly. The HSA stabilized r-tPA loading is 20.7% by weight based on total weight of solids. The % solids in the solution is 29.5%. The r-tPA loading is 12.4% by weight based on total weight of solids.

### Example 12 In Vitro Release Study of Biodegradable r-tPA Loaded DDS

Inject the triblock copolymer solution loaded with HSA stabilized r-tPA of Example 11 into a ˜6″ (150 mm) long PLGA/mPEG-b-PLGA blend biodegradable tube (0.508 mm OD and 0.356 mm ID, obtained from Isometric), incubate the tube at 37° C. for 15 minutes, let the HSA stabilized r-tPA loaded triblock copolymer solution to form a hydrogel. Cut the tube into about 10 mm sections. For each 10 mm tube section, estimated 1.14 mg of the triblock copolymer with 0.0416 mg of r-tPA loaded inside each 10 mm tube. Continue to incubate the seven sections (devices) of PLGA/mPEG-b-PLGA blend tube containing HSA stabilized r-tPA loaded triblock copolymer hydrogel at 37° C. Both ends should be sealed to prevent any loss of water before the protein release experiment starts.

The implant of 3 devices (end seals removed) of r-tPA loaded triblock copolymer hydrogel was placed in a 3 ml screw cap vial with 2 ml 1×PBS solution (pH=7.4) at 37° C. The vial was placed in a dry bath incubator at 37° C. Assay procedure: about 25% (˜0.5 ml) of the aliquots containing the solution of the released r-tPA is removed, and then add about the same amount of fresh PBS solution (already equilibrated at 37° C.), and let the release profile experiment continue. Assaying aliquots is conducted according to a pre-determined schedule. The enzyme-linked immunosorbent assays (ELISA) are used to measure the r-tPA activity and concentration, respectively. The amount of r-tPA cumulative release is determined according to the mass balance equation described in Example 4. The surface area is open enough to control the r-tPA release of 1.2 μg/day. Sustained release of r-tPA lasts over 100 days.

1. An ocular implant for the intraocular delivery of a therapeutic biologic agent, comprising:
a. a sustained-release core comprising a biodegradable gel medium, an active therapeutic biologic agent, a stabilizer; and
b. a shell of biodegradable material covering the sustained-release medium;
wherein:
the biodegradable shell material comprises poly(lactide-co-glycolide), poly(lactide-co-glycolide)-b-poly(ethylene glycol) block copolymers;
the biodegradable core gel medium comprises a B1-b-B2-b-B1 or B2-b-B1-b-B2 triblock copolymer hydrogel; and
the shell biodegradation half-life is longer than the release life of the core.

2. The ocular implant according to claim 1, wherein the implant is inserted into the vitreous body, anterior chamber, subretinal, or submacular area of an eye for the treatment of an ocular disease.

3. The ocular implant according to claim 1, wherein the implant is inserted into the vitreous body, anterior chamber, subretinal, or submacular area of an eye for the treatment of an ocular disease, and the implant dissolves at or near the site of implantation and is not removed.

4. The ocular implant according to claim 1, wherein the therapeutic biologic agent is a protein therapeutic agent.

5. The ocular implant according to claim 1, wherein the therapeutic biologic agent is a tissue plasminogen activator (tPA) therapeutic agent.

6. The ocular implant according to claim 1, wherein the therapeutic biologic agent is an anti-vascular endothelial growth factor (VEGF) therapeutic agent.

7. The ocular implant according to claim 1, wherein the release life of the therapeutic biologic agent following intraocular implantation is one day to one-year.

8. The ocular implant according to claim 1, wherein the biodegradable shell material comprises poly(lactide-co-glycolide)-b-poly(ethylene glycol)-poly(lactide-co-glycolide) triblock copolymers.

9. The ocular implant according to claim 1, wherein the biodegradable core gel sustained-release medium comprises a poly(ethylene glycol)-poly(lactide-co-glycolide)-poly(ethylene glycol) triblock copolymer hydrogel.

10. The ocular implant according to claim 1, wherein the biodegradable core gel sustained-release core-medium is selected from a group consisting of crosslinked alginate hydrogel, crosslinked hyaluronic acid, mPEG-b-PCL hydrogel, or solvent PEG-b-PLA, PLA-PEO-PLA, PEO-PLA-PEO, PLCL-b-PEO-b-PLCL, PCL-b-PEO-b-PCL, PEO-PLCL-PEO, PEO-PCL-PEO, B2-b-B1-b-B2 triblock copolymer hydrogel and mixtures thereof.

11. The ocular implant according to claim 1, wherein the stabilizer is selected from the group consisting of a pH buffer solution, an albumin, a liposome, a PEGylated liposome, and a poly(vinyl pyrrolidone) material

12. The ocular implant according to claim 2, wherein the shell has one or more perforations exposing the sustained release core to a bodily fluid in an intraocular area of the eye after implantation.

13. The ocular implant according to claim 1, wherein the shell and sustained-release core medium are substantially cylindrical in shape or substantially spherical in shape.

14. The ocular implant according to claim 1, wherein the shell completely encapsulates the core with no perforations through the shell.

15. The ocular implant according to claim 1, wherein the shell has one or more perforations.

16. The ocular implant according to claim 1, wherein the shell has a diameter from about 0.2 to about 1.0 mm and a length from about 2 to about 12 mm.

17. The ocular implant according to claim 1, wherein the shell has a diameter of about 0.5 mm and a length of about 7 mm.

18. An ocular implant for the intraocoluar delivery of a therapeutic biologic agent, comprising:
a. a sustained-release core comprising a biodegradable gel medium, an active therapeutic biologic agent, a biologic stabilizer; and
b. a shell of biodegradable material covering the sustained-release medium;
wherein:
the biodegradable shell material comprises poly(lactide-co-glycolide), poly(lactide-co-glycolide)-b-poly(ethylene glycol) block copolymers;
the biodegradable core gel medium comprises a B1-b-B2-b-B1 or B2-b-B1-b-B2 triblock copolymer hydrogel;
the shell biodegradation half-life is longer than the release life of the core;
the shell has one or more perforations exposing the sustained release core to a bodily fluid
the therapeutic biologic agent is a protein therapeutic;
the therapeutic biologic agent is released over a period of time varying from one day to one year;
wherein the implant is fully resorbed and is not removed.

19. A method of treating a disease of the eye, comprising an ocular implant for the intravitreal delivery of a therapeutic biologic agent according to claim 1.

20. The method of claim 18, wherein the disease is selected from neovascularization in the back of the eye, (e.g. age-related macular degeneration, diabetic retinopathy, or retinal vein occlusions), or glaucoma-related ocular disease (e.g. open, closed-angle, or other forms).
